SEQUENCE LISTING (4 sequences)

SEQ ID NO: 1 - protein, 14 aa, Homo sapiens
SDRSADPSNQTGIS

SEQ ID NO: 2 - protein, 17 aa, Homo sapiens
GLYSDRSADPSNQTGIS

SEQ ID NO: 3 - protein, 14 aa, Homo sapiens
CDRSADPSNQTGIC

SEQ ID NO: 4 - protein, 17 aa, Homo sapiens
GLYCDRSADPSNQTGIC

(19) United States
(12) Patent Application Publication
(10) Pub. No.: US 2026/0364140 A1
(43) Pub. Date: Nov. 19, 2026

(54) CCN3-DERRIVED THERAPEUTIC PEPTIDE FOR TREATING LUNG FIBROSIS, IDIOPATHIC PULMONARY FIBROSIS, AND METHODS OF USE

(71) Applicant: BLR Bio, LLC, Kenosha, WI (US)

(72) Inventor: Bruce L. Riser, Kenosha, WI (US)

(21) Appl. No.: 19/654,061

(22) Filed: Apr. 21, 2026

Related U.S. Application Data
(60) Provisional application No. 63/792,851, filed on Apr. 22, 2025.

(51) Int. Cl. A61K 38/10; A61K 9/00; A61P 11/00; G01N 33/68

(52) U.S. Cl. CPC A61K 38/10; A61K 9/0019; A61P 11/00; G01N 33/6884; G01N 2333/4703; G01N 2333/4704; G01N 2333/475; G01N 2333/495; G01N 2333/522; G01N 2333/525; G01N 2333/54; G01N 2333/545; G01N 2333/70546; G01N 2333/78; G01N 2333/90245; G01N 2333/96494; G01N 2800/12; G01N 2800/52; G01N 2800/56

(57) ABSTRACT

Provided herein are methods and compositions for CCN3-like peptide for treating lung fibrosis, Idiopathic Pulmonary Fibrosis, Interstitial Lung Disease, and associated conditions.

### CROSS-REFERENCE TO RELATED APPLICATIONS

The present application claims priority to U.S. provisional application Ser. No. 63/792,851, filed Apr. 22, 2025, herein incorporated by reference in its entirety.

### SEQUENCE LISTING

The present application contains a Sequence Listing which is submitted electronically in XML format and is incorporated by reference in its entirety. Said XML file was created on Aug. 4, 2026, is named XML_Sequence_BLR103US.xml and 4,537 bytes in size.

### BACKGROUND

The invention generally relates to treating fibrotic conditions, and more specifically, to treating lung fibro-inflammatory disease.

Interstitial lung disease (ILD) is recognized as a group of diseases characterized by a combination of (a) chronic inflammation within the lung, consisting of an accumulation of chronic inflammatory cells (predominantly lymphocytes and macrophages) and increased levels of numerous pro-inflammatory cytokines, chemokines, and cell surface molecules; and (b) varying degrees of lung fibrosis. As shown in FIG. 1, (O Kalchiem-Dekel, et al., J Clinical Medicine 2018), any particular form of ILD may manifest primarily as an inflammatory lung disease, with little if any features of fibrosis and a generally favorable response to anti-inflammatory or immunosuppressive treatments. Second, it may manifest primarily as a fibrotic pulmonary disease, with a generally poor prognosis and limited efficacious therapies. Third it may manifest with varying degrees of inflammation and fibrosis (Kalchiem-Dekel et al., 2018). The one exception is idiopathic (meaning the cause is unknown) pulmonary fibrosis (IPF). IPF is considered a prototypical fibrotic lung disease and is the largest specific group within the overall pulmonary fibrosis group (FIG. 1), with anti-inflammatory or immunosuppressive treatments being ineffective and at times harmful. The term fibrosis generally implies histologically an excess deposition of collagen, but there have been conflicting reports over many years as to the precise histopathologic components of clinically recognized pulmonary fibrosis (Kalchiem-Dekel et al., 2018). Despite this uncertainty, pulmonary fibrosis can reliably be recognized on CT imaging of the chest by its characteristic findings. IPF is a devastating chronic lung disease, primarily affecting middle aged and older adults. Lung function decline is gradual, with the potential for intermittent, unpredictable, acute exacerbations and the development of associated pulmonary hypertension.

Disease pathogenesis remains elusive and controversial. Currently, the prevailing hypothesis assumes an ineffective wound healing response to alveolar epithelial cell injury. Injury magnitude and susceptibility appear to be related to aging and genetic predisposition, with subsequent innate immune system and fibroblast activation. The overall prognosis of patients with IPF is highly unpredictable and poor with median survival after diagnosis being approximately 3.8 years. Attempts to understand disease pathogenesis, identify prognosticators, and unravel novel therapeutic targets have relied on animal models. Unfortunately, no animal model fully recapitulates exactly the histological pattern of IPF or exhibits features of progressive disease. However, animal models are essential prerequisites for the subsequent application of prognostic tests and therapeutic interventions. Numerous clinical trials have been completed based on preclinical studies in animals and have led to the FDA approval of three drugs, Pirfenidone (Esbriet) and Nintedanib (Ofev) and very recently (2025) Nerandomilast (Jascyd). Although these drugs have been shown to slow progressive loss in lung function (i.e. forced vital lung capacity), they do not cure IPF, thus at the best-case leaving patients with significant pulmonary disability. While no animal model perfectly recapitulates the human IP, the intratracheal bleomycin (BLM) model is the best-characterized animal model available for preclinical testing (Tashiro et al., 2017).

The present application attempts to solve these problems, as well as others.

### SUMMARY OF THE INVENTION

Provided herein are methods and compositions for CCN3-derived peptide for treating lung fibrosis, Interstitial Lung Disease, Idiopathic Pulmonary Fibrosis, and associated conditions.

The methods and composition are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a schematic overview of interstitial lung disease (ILD) and pulmonary fibrosis, which includes six ILDs. Other less common ILDs would occupy the remaining space within the ILD and fibrosis ovals and would manifest in varying degrees of inflammation and/or fibrosis depending on the particular disorder.

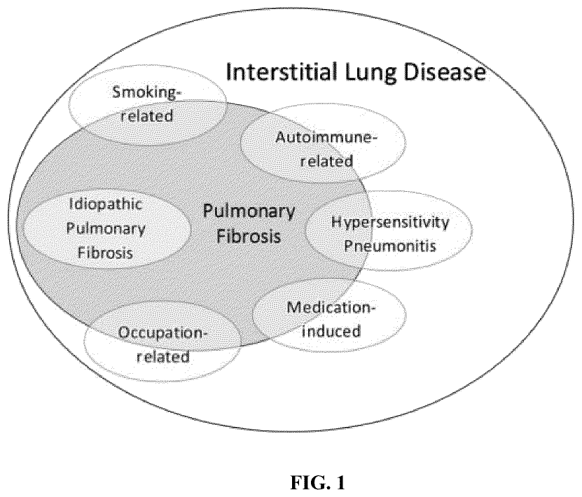

FIG. 2 shows BLR-200 treatment (3× per week) blocked the development of interstitial lung disease (ILD) induced by 21 days of bleomycin exposure. Histopathology by both H&E (upper right figures) and Trichrome staining (lower right figures) showed marked collagen deposition in the diseased group (BLM treated) with the negative control peptide (BLR 200 sequence but with amino acids in scrambled positions) appearing very much like the control non diseased animals. Semi-quantitative assessment by the Ashcroft scoring method confirmed that animals treated with bleomycin and the scrambled peptide showed marked fibrosis whereas the bleomycin plus BLR-200 animals were only very slightly increased from normal.

FIG. 3 are graphs shows that intratracheal administration of bleomycin for 21 days resulted in the increase in key elements (and markers) of fibrosis-lung collagen levels, plasma TGF-b1, plasma metalloproteinase (MMP) 7 levels, and bronchoalveolar lavage (BAL) fluid MMP 7 levels-all increases were completely neutralized by BLR-200 treatment 3×/week.

FIG. 4 are lung sections stained with hematoxylin and eosin, and Ashcroft lung architecture scores were calculated. In addition, lung collagen levels were assessed using a colorimetric hydroxyproline assay. Finally, real-time polymerase chain reaction assay was conducted to assess fibronectin-1 (FN-1) and WSIP-1 (CCN4) mRNA expression levels. All measurements were elevated and all were neutralized, or very nearly neutralized by BLR-200 treatment.

FIG. 5 shows a clear increase in lung Collagen content (the overproduction of collagens type 1 and type 3 are characteristic of lung fibrosis), assessed here using a colorimetric hydroxyproline assay); there was a very clear normalization of collagen levels by BLR-200 treatment 3×/week.

FIG. 6 shows a mild increase in TGF-b1 levels with disease (TGFb-1 can stimulate a matrix-preserving transcriptional program in fibroblasts by activating Smad-dependent and non-Smad dependent pathways levels driving increased matrix deposition); BLR-200 treatment had a small lowering effect on TGF-b1.

FIG. 7 shows an increase in plasma MMP-7 (matrix metalloproteinase-7 is involved in the development and progression of fibrosis by influencing inflammation, innate immunity, apoptosis, and fibroproliferation); levels increased with disease; BLR-200 had a small lowering effect of MMP-7 levels in plasma.

FIG. 8 shows the bronchial alveolar lavage (BAL) fluid MMP-7 levels are increased (but not in all animals); all BLR-200 animals treated had normal healthy levels.

FIG. 9 shows plasminogen activator inhibitor-1 (PAI-1) (a protein implicated in the pathology of fibrosis in lungs) lung mRNA levels; levels appear increased in IPF, (but not all animals); BLR-200 treated animals all had the levels of the normal healthy mice.

FIG. 10 shows the measurement of CCN2 levels (a profibrotic protein in some forms of fibrosis and thought to be a target of BLR-200) lung mRNA levels; surprisingly, CCN2 levels did not increase in the IPF fibrosis and remain unaffected by BLR-200.

FIG. 11 shows TNC (tenasin c, an ECM glycoprotein that plays a crucial role in development, tissue repair, and disease including in IPF and in chronic lung disease) lung mRNA levels; TNC levels here increased markedly and were normalized by BLR-200 treatment.

FIG. 12 shows IL-33 (a cytokine involved in cell-to-cell communication within the immune system is released during tissue damage or stress to alert the immune system) lung mRNA levels; IL-33 levels were increased with IPF and appeared lowered by treatment with BLR-200.

FIG. 13 shows CCN1 (a profibrotic matricellular protein in the CCN family) mRNA levels levels; levels were unchanged in the model of IPF and there is no effect of BLR-200.

FIG. 14 shows SOX2 (a key molecule in lung development, stem cell function, and repair processes, influencing airway cell fate, plasticity, and regeneration); SOX2 levels are unchanged in the model of IPF and there is no effect of BLR-200.

FIG. 15 shows ITGA11, also known as integrin subunit alpha 11 (plays a significant role in tissue fibrosis, particularly in organs like the liver, lungs, and kidneys and is selectively overexpressed in myofibroblasts during fibrotic processes); ITGA11 decreased in the IPF model, and this is somewhat normalized by BLR-200.

FIG. 16 shows thrombospondin-1 (THBS1), also known as TSP-1 (a protein involved in various biological processes, particularly in inflammation, tissue repair, and fibrosis and activates TGF-b1; THBS1 appears slightly increased in IPF but not in BLR-200 treated.

FIG. 17 shows ACTA2 is not affected in IPF nor by BLR-200.

FIG. 18 shows TNF-alpha (tumor necrosis factor alpha) is a cytokine that plays a crucial role in the immune system and is involved in both acute and chronic inflammation); TNF-alpha expression is increased in IPF and normalized by BLR-200.

FIG. 19 shows IL1beta (interleukin-1 beta is a pro-inflammatory cytokine involved in the body's immune response to infection and injury); IL1b expression is not affected by IPF nor the drug.

FIG. 20 shows PLOD2 (procollagen-lysine, 2-oxoglutarate 5-dioxygenase 2, an enzyme involved in collagen cross-linking, that is upregulated in fibrotic tissues and contributes to matrix stiffening and collagen stabilization; PLOD2 expression was surprisingly lowered in IPF and not affected by the drug.

FIG. 21 shows CCN4 (also known as WISP1, thought to play a role in both promoting and preventing tissue fibrosis); CCN4 expression was elevated slightly in IPF and normalized by drug treatment.

FIG. 22 shows IL-17a (a pro-fibrotic cytokine that drives lung fibrosis by promoting inflammation, fibroblast proliferation, and myofibroblast differentiation, primarily through TGF-b dependent pathway expression) lung mRNA levels; IL-17a levels were not elevated in IPF and not affected by drug treatment.

FIG. 23 shows Fibronectin-1 (FN-1), a matrix glycoprotein involved in a variety of cellular processes, including cell adhesion, migration, and the organization of the extracellular matrix and is implicated in the development of various diseases, including cancer, fibrosis, and cardiovascular disease); expression of FN-1 lung mRNA levels were elevated in disease and normalized by BLR-200.

FIG. 24 shows IL-23 (a pro-fibrotic cytokine that is thought to drive fibrosis including IPF and autoimmune disease) lung mRNA levels; IL23 lung mRNA expression levels were not affected by IPF nor the drug treatment.

FIG. 25 shows COL3A (crucial for the strength and support of various tissues, including skin, lungs, intestinal walls, and blood vessels) lung mRNA expression; COL3A expression was increased in IPF and nearly normalized by BLR-200 (elevated in 1 treated animal).

FIG. 26 shows Endothelin-1 (EDN1) (promotes myofibroblast induction through the ETA receptor via a Rac/phosphoinositide 3-kinase/Akt-dependent pathway and is essential for the enhanced contractile phenotype of fibrotic fibroblasts); Endothelin-1 expression was not elevated by disease was not affected by the drug.

FIG. 27 shows CXCL2 (when overexpressed is associated with exacerbated lung fibrosis in animals) lung mRNA levels; levels were increased in some of the IPF animals, and none of the BLR-200 treated animals.

FIG. 28 shows CCN3 (known to be a natural antifibrotic in several types of fibrosis); expression was unchanged in IPF and in the BLR-200 treated mice group.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes, or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENTS

### CCNs and BLR-200.

The mechanosensitive transcriptional cofactor yes-associated protein (YAP)-1 is essential for fibrogenesis, including maintenance of the persistent myofibroblast phenotype in Systemic sclerosis (SSc), often referred to as scleroderma, (Shi-wen et al., 2021). Prototypical YAP1 targets are the pro-adhesive matricellular proteins Central Communication Network (CCN) proteins CCN1 and CCN2, critical profibrotic modulators (Riser et al., 2015; Tsang et al., 2020a; Tsang & Leask, 2015). In a fibrotic context, both are significantly upregulated in SSc skin fibrosis; CCN2 is crucial for the myofibroblast differentiation, whereas CCN1 is crucial for ECM stability (Hutchenreuther et al., 2024; Liu et al., 2011; Tsang et al., 2020b). The related protein CCN3 has been shown to be anti-fibrotic and works in a yin/yang manner with CCN2 (and perhaps CCN1) to re-create homeostasis in models of kidney fibrosis (Riser et al., 2009, 2014). The role of CCN1 and CCN2 has not been clearly established in fibro-inflammatory lung diseases like ILD (including that in SSc) and IPF (Ganzieben et al, 2025 iScience). Small CCN3-derived peptides were created, that include BLR-200, to mimic, and improve on, CCN3's specific ability to treat fibrosis. BLR-200 (as disclosed in U.S. Pat. Nos. 9,114,112, 10,351,608, 11,505,582, 12,024,544, herein incorporated by reference in their entireties), blocks the activation of a specific fibroblast subtype necessary for myofibroblast differentiation in SSc skin, however the peptide(s) role in fibro-inflammatory lung disease is unknown. Currently, no single drug treats all forms of fibrosis, as the underlying causes and mechanisms vary across different types and organs. Instead, treatments often target specific types or organs, with medications like pirfenidone and nintedanib approved for idiopathic pulmonary fibrosis (IPF). Neither of these drugs, while used in IPF are efficacious in treating SSc skin fibrosis and make it unlikely to have a single drug that can cross over to many forms of fibrosis.

### BLR-200 Efficacy in Lung Fibrosis.

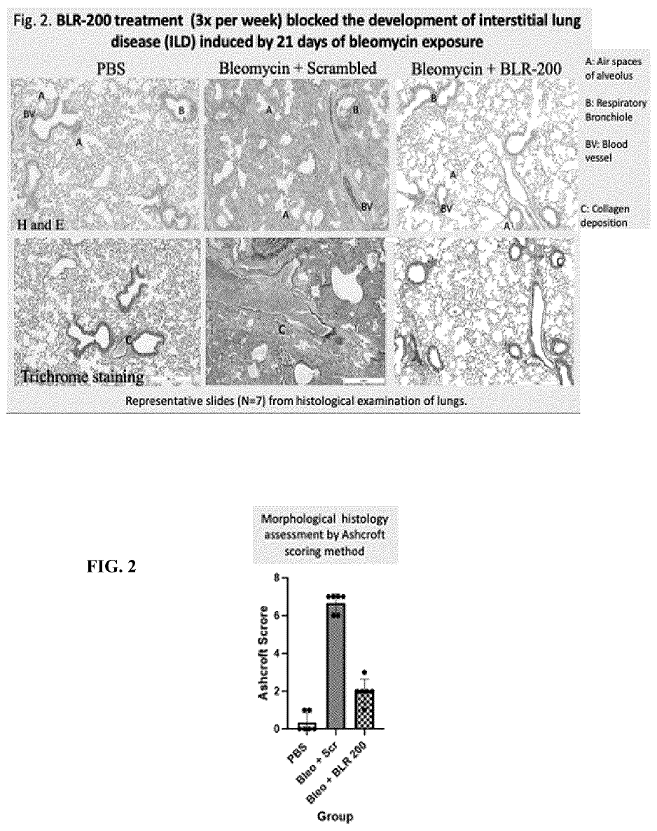

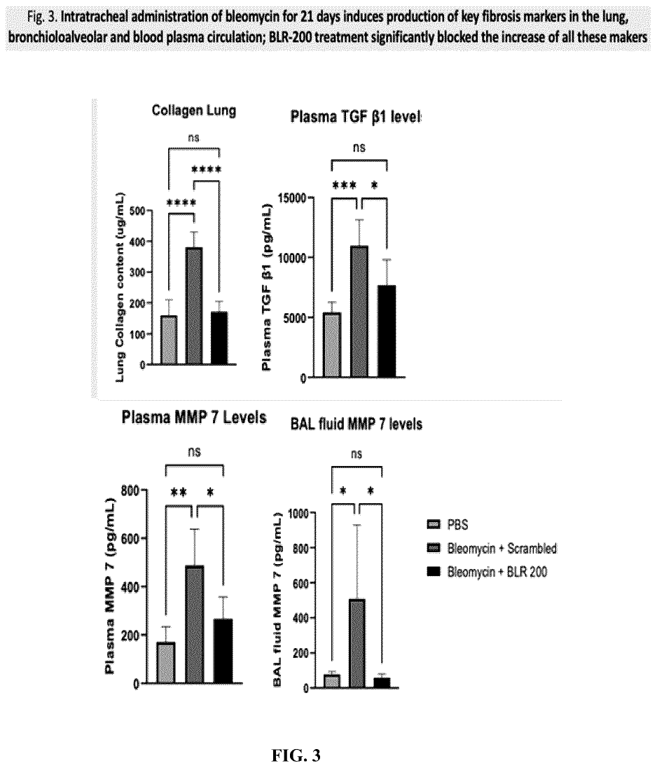

The aim was to determine the potential efficacy of BLR-200 in treating human fibro-inflammatory lung disease including ILD, IPF, including that in patients with SSc. For this we used the bleomycin model of lung inflammation and fibrosis; bleomycin is delivered to the lungs via an intratracheal administration (IT) daily for 21 days. This is a well characterized model exhibiting pathogenesis like that seen it human IPF (Hu et al., 2015). As a placebo, one group was treated with a scrambled animo acid sequence (negative drug control) peptide (beginning one day after the first bleomycin dose, ie when inflammation is already ongoing), then three times per week delivered by the intraperitoneal (IP) route. We have identified in comparative mouse pharmacokinetic (PK) studies that BLR-200 administered via the IP route creates a PK profile that is very similar to that delivered in by the subcutaneous (SC) route. This is important since the SC route is an acceptable route for human patients. IP was used here because it is more accessible in mice than SC. BLR-200 was administered by the same route and on the same schedule as the scrambled peptide described above. After 21 days the lungs, blood, and bronchioalveolar (BAL) washes were collected for analysis. As expected, results from the histological analysis showed that lungs from animals receiving bleomycin plus the control, scrambled peptide (that is animals with IPF-like disease) had much reduced airways along with a highly increased collagen deposition, both characteristic of IPF lung fibrosis including that in SSc (FIG. 2). Remarkably, the lungs from the animals receiving bleomycin then BLR-200 treatment 3× per week resembled the PBS treated healthy control animals. That is, the BLR-200 drug blocked/reversed the development of disease measured by these parameters including the Ashcroft semi-quantitative scoring of pulmonary fibrosis (FIG. 2). As additional determinates, key elements and markers of inflammation and fibrosis, including collagen deposition (by quantitative hydroxyproline analysis) were examined along with markers of fibrosis in plasma and bronchoalveolar (BAL) washes (FIG. 3). Circulating TGF-b1 and MMP-7, as well BAL MMP-7 and collagen were all increased with the disease and normalized by BLR-200 treatment (FIG. 3). Increased collagen is endpoint commonly characterizing fibrosis, whereas TGF-b1 is a known causal factor driving some forms of fibrosis, or at some periods in the development of fibrosis; both were increased in the animal group with disease and neutralized or greatly reduced by BLR-200 drug treatment. MMP-7 (metalloproteinase 7) is involved in the turnover of collagen and is increased in fibrosis and IPF (as observed here) and was also greatly reduced or neutralized decreased by BLR-200 treatment in both lung BAL fluids (washings of the lung) and in blood plasma.

No previous studies have shown an effect of BLR-200, or any peptide from this region of CCN3 on lung fibroinflammatory disease including IPF. Neither have any studies examined effect of BLR-20 or similar sequences on the levels or activity of the CCN4 a matricellular member of the CCN family that appears to be pro-fibrotic in a limited number of organ fibrotic diseases (not e.g in skin fibrosis) including that in the lung. The finding here that it was increased in disease, was expected but the ability of BLR-200 to neutralize this increase was an unexpected finding.

The diagnostic system for IPF/SSc lung fibro-inflammatory disease, comprises: determining the stage of the IPF/SSc lung fibrosis, and the early response to therapy by measuring the change in response to BLR-200 and/or other antifibrotic, or potential anti-fibrotic treatments on levels of markers, wherein the level of markers are obtained from at least one source including blood/plasma, lung tissue, or bronchiolar washings; wherein the change in measurement is selected from the group consisting of: COL1A, COL3a, CCN1, CCN2, CCN3, CCN4, CXCL2, EDN1, FN1, IL-23, IL17A, PLOD2, IL-beta, TNF-alpha, ACTA2, THBS1, ITGA1, SOX2, IL33, TNC, PAI-1, MMP7, TGF-b1. Any or all the above measurements could also be used in combination with more standard assessments of diseases e.g. chest scans.

BLR-200 is based on the structure/potential function of the so called IGFBP-like domain (the domain contains amino acid sequences similar to a part of insulin growth factor binding proteins and with the amino acid sequence of SEQ ID NO. 1: SDRSADPSNQTGIS), or structurally related sequences within the entire IGFBP-like domain, including, but not limited to SEQ ID NO. 2: GLYSDRSADPSNQTGIS, or SEQ ID NO. 2: CDRSADPSNQTGIC, or SEQ ID NO. 4: GLYCDRSADPSNQTGIC, or a recombinant CCN3 protein containing the IGFBP domain with all peptides being either the native sequence, or with one or more amino acid substitutions where e.g. the cysteines substituted by serine.

As supported by the following examples, a method of treating a patient with Idiopathic Pulmonary Fibrosis (IPF) and/or related lung fibro-inflammatory disease, including Interstitial Lung Disease, comprising the steps of: administering an effective dose of a synthetic peptide based on: 1) the IGFBP-like domain (insulin-like growth factor binding protein) contained in the CCN3 protein (Cellular Communication Network protein) or, 2) BLR-200 that is based on the structure/function of the IGFBP-like domain and with the amino acid sequence of SEQ ID NO. 1: SDRSADPSNQTGIS), or 3) structurally related sequences within the entire IGFBP-like domain, including SEQ ID NO. 2: GLYSDRSADPSNQTGIS, or SEQ ID NO. 2: CDRSADPSNQTGIC, or SEQ ID NO. 4: GLYCDRSADPSNQTGIC, 4) or a recombinant CCN3 protein containing the IGFBP domain-all peptides being either the native sequence, or with one or more amino acid substitutions where e.g. the cysteines substituted by serine. In one embodiment, the patient is not diagnosed with IPF but has predisposition for developing IPF or lung fibrosis, to prevent IPF or lung fibrosis. In one embodiment, the synthetic peptide is incorporated into a kit formulated in saline or phosphate balanced saline (PBS), or other such carrier, or the same freeze dried and in a vial that can be reconstituted in water or placed into a prefilled syringe, and then administered to a patient by a IV, IP, SC, IM injection, or in an oral delivery form, or into the lungs as an inhaled suspension via any of many lung delivery devices. In one embodiment, a formulation is loaded into a device that is either implanted in the patient or is attached to the outside of the body and is designed to administer the drug. In one embodiment, the drug is administered from about 1 time per week to about 4 times per week. In one embodiment, the drug dose administered is between about 0.081 and about 1.62 microgram (ug)/kg (based on the conversion to human of a mouse effective dose of between about 1 ug/kg and about 20 ug/kg. In one embodiment, the drug dose is adjusted based on determination of the stage or condition of fibro-inflammatory progression/regression. In one embodiment, the determination of drug dose is by the measurement of serum, tissue/body fluid, bronchial washings of biomarkers/markers, or CT scan MRI or similar diagnostic for fibro-inflammatory disease/fibrosis, and/or stage of IPF or SSc. In one embodiment, the patient is diagnosed with IPF or is developing IPF or lung fibrosis, to prevent IPF or lung fibrosis.

As supported by the following examples, a diagnostic system for IPF/SSc lung fibro-inflammatory disease, comprises: determining the stage of the IPF/SSc lung fibrosis and the early response to therapy by using measuring the change in response to BLR-200 in levels of markers, wherein the level of markers are obtained from at least on source including blood/plasma, lung tissue, or bronchiolar washings; wherein the change in measurement is selected from the group consisting of: COL1A, COL3a, CCN1, CCN2, CCN3, CCN4, CXCL2, EDN1, FN1, IL-23, IL17A, PLOD2, IL-beta, TNF-alpha, ACTA2, THBS1, ITGA1, SOX2, IL33, TNC, PAI-1, MMP7, TGF-b1. In one embodiment, the overall stage determined can be used to adjust the level of medication to obtain the optimum response in the patient. In one embodiment, these measurements can by combined with CT scan MRI or similar to obtain a more accurate diagnostic and prognostic profile for fibro-inflammatory disease/fibrosis, and/or stage of IPF or SSc.

The term “administration” or “administering” refers to a method of providing a dosage of a compound or active ingredient or composition to a subject, introducing a predetermined substance into an individual using any suitable method, and the mode of administration may be any of the modes commonly used in the art, for example, the substance may be administered by oral, skin, intravenous, intramuscular, subcutaneous routes or the like, and preferably, it can be administered by an oral route. Modes of administration, dosing schedules disclosed compounds and compositions can be determined according to the criteria generally taken into account in the establishment of a metabolic disorder treatment adapted to, for example, a patient's metabolic pattern. The compositions can be administered such that they conditioned to be treated before a metabolic disorder occurs.

In the present disclosure, an “effective amount” or an “effective dose” of the composition, or composition refers to an amount of composition that, once administered to a subject, will reach the subject's bloodstream and/or bodily tissues and achieve the effect disclosed herein. It will be understood, however, that the total daily usage may be decided by the attending physician within the scope of sound medical judgment. The specific therapeutically effective dose level for any particular subject will depend upon a variety of factors including the disorder being treated and the severity of the disorder; the specific composition employed, the age, body weight, general health, sex and diet of the subject; the time of administration, route of administration, and rate of excretion of ergothioneine employed; the duration of the treatment; drugs used in combination or coincidental with active compounds disclosed herein; and like factors well known in the medical arts. For example, it is well known within the skill of the art to start doses of the compound at levels lower than those required to achieve the desired therapeutic effect and to gradually increase the dosage until the desired effect is achieved. In addition, a “therapeutically effective amount” is the amount that will elicit the biological or medical response of a tissue, system, or subject that is being sought by a researcher or clinician, and in particular elicit some desired therapeutic or prophylactic effect for extending lifespan or improving longevity in a mammal. The term effective amount may include or be synonymous with a pharmaceutically effective amount or a therapeutically effective amount.

The term “improving”, “reducing” or “treating” are used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

“Treatment” or “treating” includes (1) inhibiting a condition in a subject or patient experiencing or displaying the pathology or symptomatology of the disease (e.g., arresting further development of the pathology and/or symptomatology), (2) ameliorating a metabolic condition in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

As used herein, the term “patient” or “subject” refers to a living mammalian organism, such as a human, monkey, cow, sheep, goat, dog, cat, mouse, rat, guinea pig, or transgenic non-human species thereof. In certain embodiments, the patient or subject is a primate. Non-limiting examples of human patients are adults, juveniles, and infants.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### Example: (CCN3)-Based Peptide BLR200 Impairs Bleomycin-Induced Lung Fibrosis

Animals-C57 BL/6 J mice (Male); Age-6 weeks; Dose: 10 μg/kg.

Six-week-old mice (6 per group) were subjected to the bleomycin-induced (single tracheal injection of 1.5 U/kg) mode of lung fibrosis. Mice were then injected intraperitoneally the following day with control scrambled peptide or BLR-200 (10 microgram/kg) every other day for 21 days. Blood, BAL fluid and Lung collection was at Day 22 when mice were euthanized.

TABLE

| | Number of animals | Number of animals |
|---|---|---|
| Group | injected | survived |
| PBS | 6 | 6 |
| Bleomycin + Scrambled | 10 | 8 |
| Bleomycin + BLR 200 | 10 | 7 |

Lung sections were stained with hematoxylin and eosin, and Ashcroft lung architecture scores were calculated. In addition, lung collagen levels were assessed using a colorimetric hydroxyproline assay. Finally, real-time polymerase chain reaction assay was conducted to assess fibronectin-1 (FN-1) and WSIP-1 (CCN4) mRNA expression.

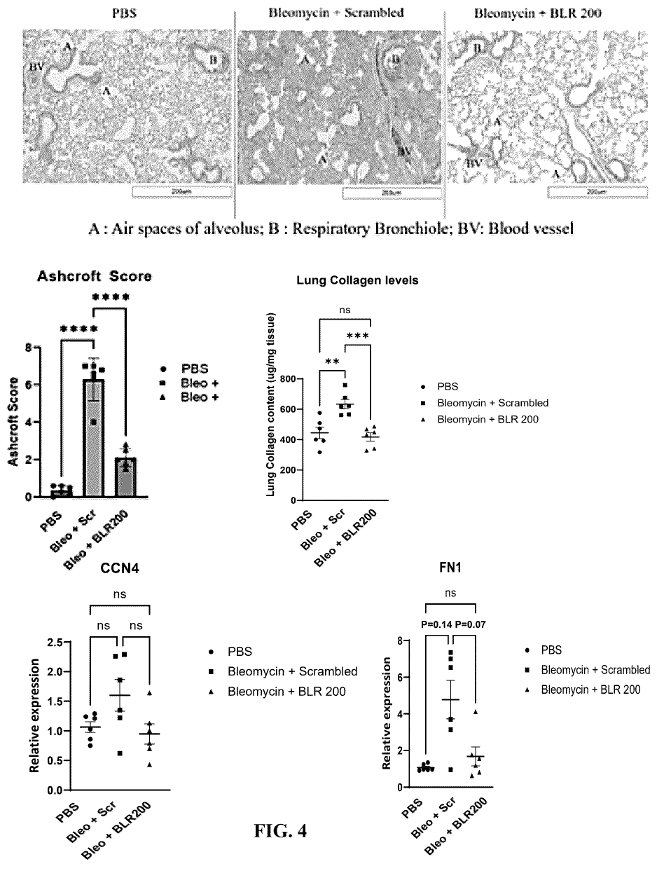

Bleomycin treatment caused an increase in lung fibrosis as shown in FIG. 4 (Ashcroft score, collagen levels, FN-1 and CCN4 mRNA expression). Histology shows that animals receiving bleomycin plus the control, scrambled peptide had much reduced airways along with a highly increased collagen deposition, both characteristic of IPF lung fibrosis.

Exposure to BLR-200 prevented/treated the bleomycin-induced increase in fibrosis (Ashcroft score, collagen levels and both FN-1 and CCN4 mRNA expression, as shown in FIG. 4).

BLR-200 comprises a novel treatment to block or reverse scleroderma/SSc-associated lung fibrosis and or non SSc idiopathic pulmonary fibrosis or fibro-inflammatory pulmonary disease in

### Example: (CCN3)-Based Peptide BLR-200 Selectively Neutralizes Key Elements and Markers of Progression in a Model of Human Bleomycin-Induced IPF/Lung Fibro-Inflammatory Disease

All data that follows are measurements of mRNA levels from the lungs of mice using RT-qPCR, to determine changes in additional key elements and markers of fibrosis progression as a method of determining the changes of these elements and markers with the formation of IPF/fibroinflammatory disease and the effect of BLR-200 treatment.

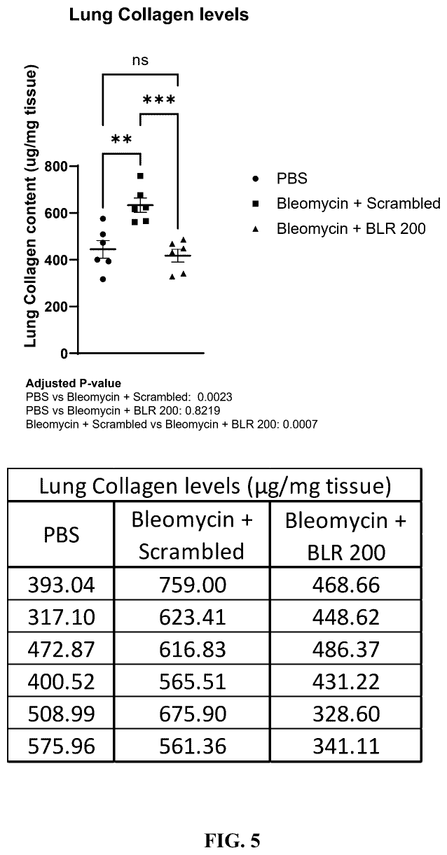

FIG. 5 shows a clear increase in lung Collagen content with disease; and very clear normalization by BLR-200.

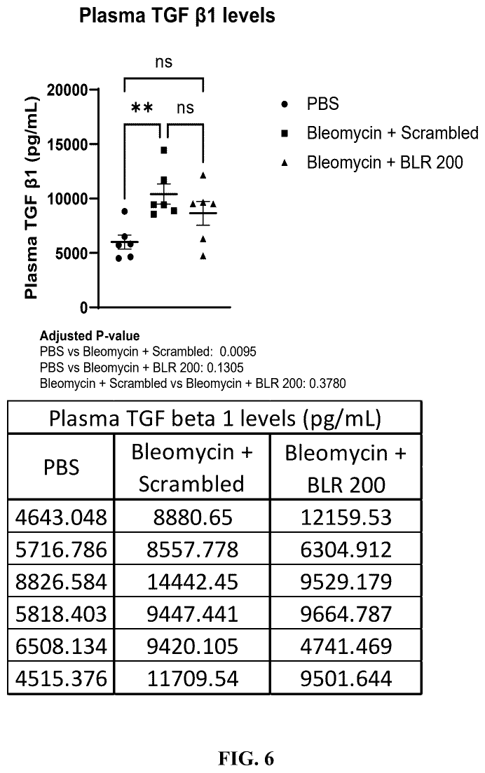

FIG. 6 is a clear increase in TGF-b1 levels with disease; (TGF-b1 stimulates a matrix-preserving transcriptional program in fibroblasts by activating Smad-dependent and non-Smad pathways) levels with disease; BLR200 might have a small lowering effect.

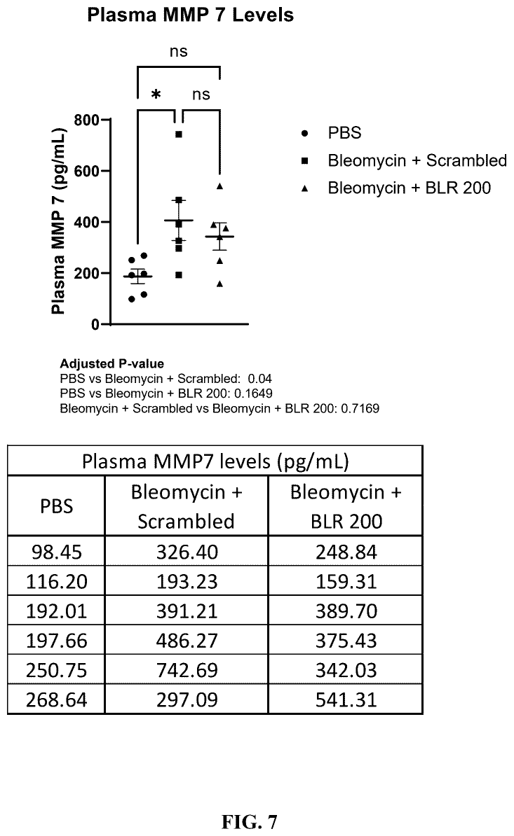

FIG. 7 shows an increase in plasma MMP-7 (matrix metalloproteinase-7 is involved in the development and progression of fibrosis by influencing inflammation, innate immunity, apoptosis, and fibroproliferation) levels increased with disease; there may be a small lowering effect of BLR-200 in plasma.

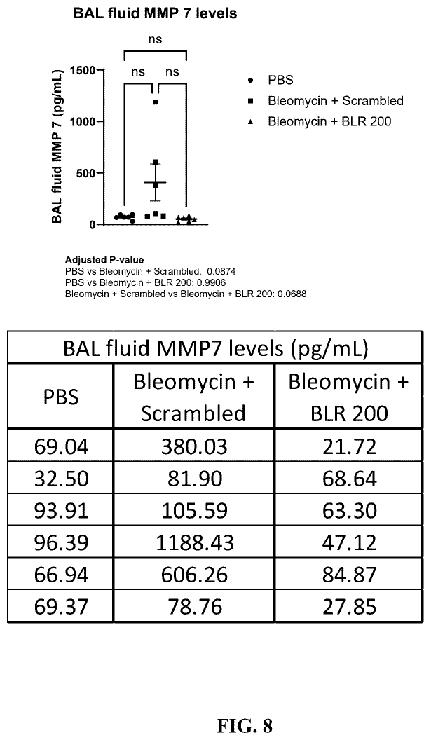

FIG. 8 is showing the BAL fluid MMP-7 levels are increased in the disease group (but not in all animals) and reduced by BLR-200.

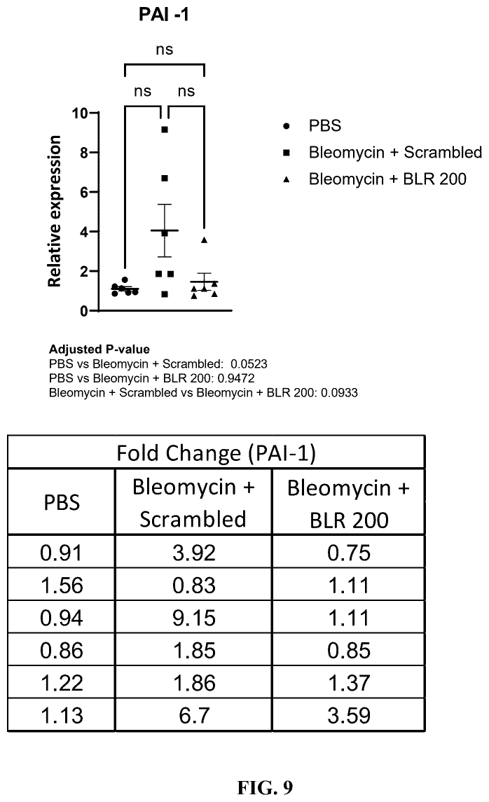

FIG. 9 is showing PAI-1 (implicated in the pathology of fibrosis in lung) lung mRNA levels appear increased in IPF and reduced in all but 1 by BLR-200 treatment.

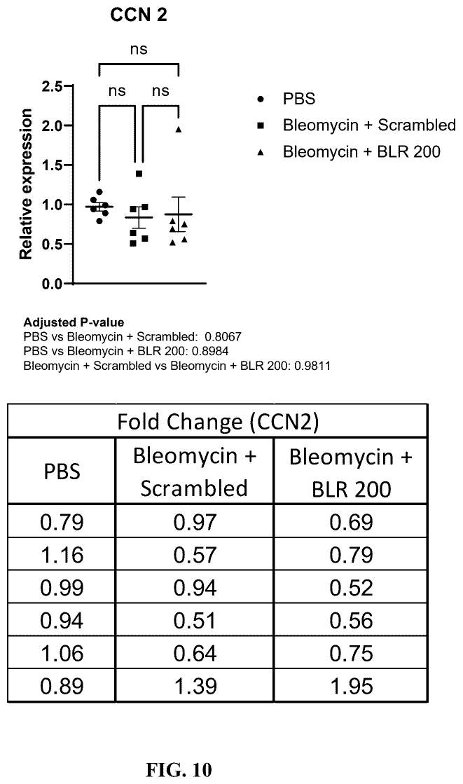

FIG. 10 showing CCN2 (a target of BLR-200) lung mRNA levels unexpectedly, do not increase in IPF fibrosis, and were not affected by BLR-200.

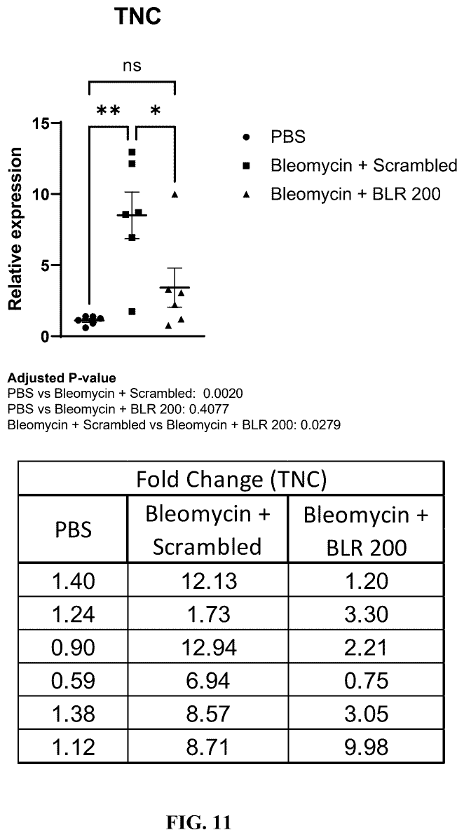

FIG. 11 is showing TNC (tenasin C, an ECM glycoprotein that plays a crucial role in development, tissue repair, and disease) lung mRNA levels uniquely increase in IPF fibrosis and are largely normalized by BLR-200 treatment.

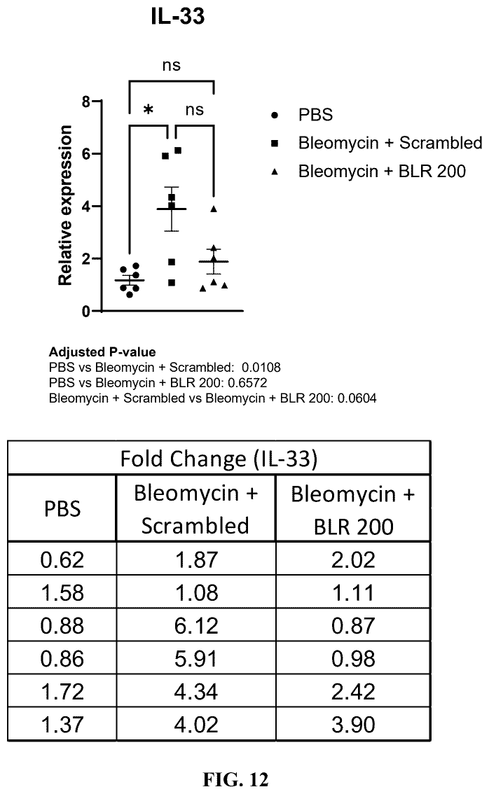

FIG. 12 is showing IL-33 (a cytokine involved in cell-to-cell communication within the immune system is released during tissue damage or stress to alert the immune system) lung mRNA levels are increased with IPF and lowered by treatment with BLR-200.

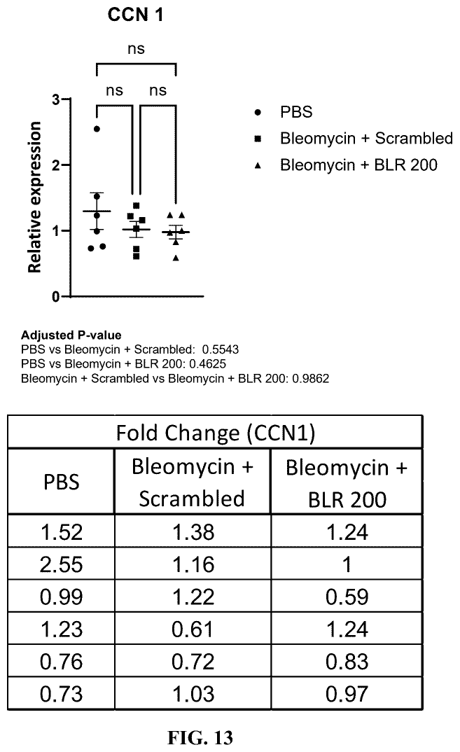

FIG. 13 is showing CCN1 (a profibrotic matricellular protein) lung mRNA levels are unexpectedly unchanged in the model of IPF, and there is no effect of BLR-200.

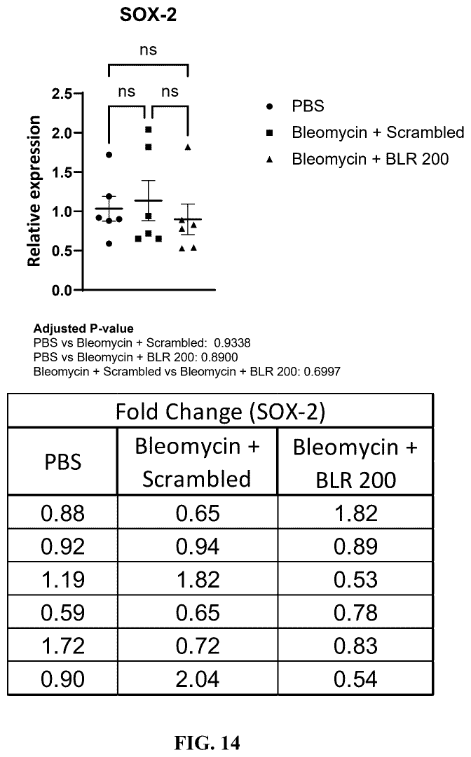

FIG. 14 is showing SOX2 (a key player in lung development, stem cell function, and repair processes, influencing airway cell fate, plasticity, and regeneration) lung mRNA levels are unchanged in the model of IPF and there is no effect of BLR-200.

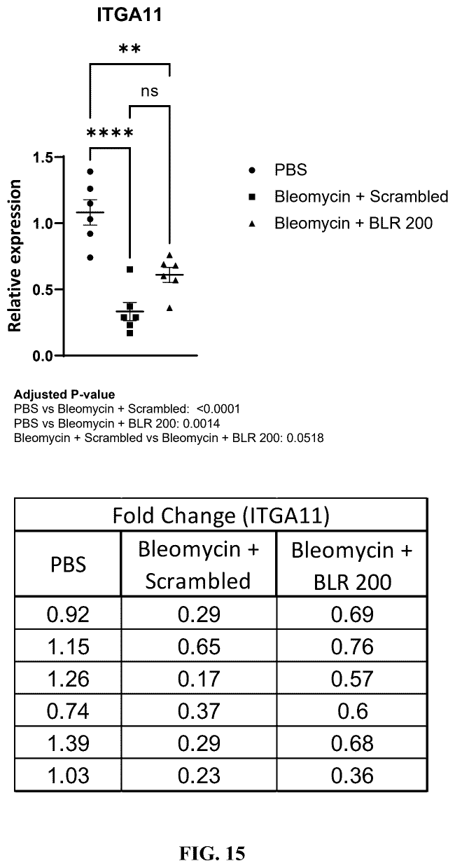

FIG. 15 is showing ITGA11 (known as integrin subunit alpha 11, that plays a significant role in tissue fibrosis, particularly in organs like the liver, lungs, and kidneys and selectively overexpressed in myofibroblasts during fibrotic processes) lung mRNA levels decrease in the IPF model, and this effect may be somewhat neutralized by BLR-200.

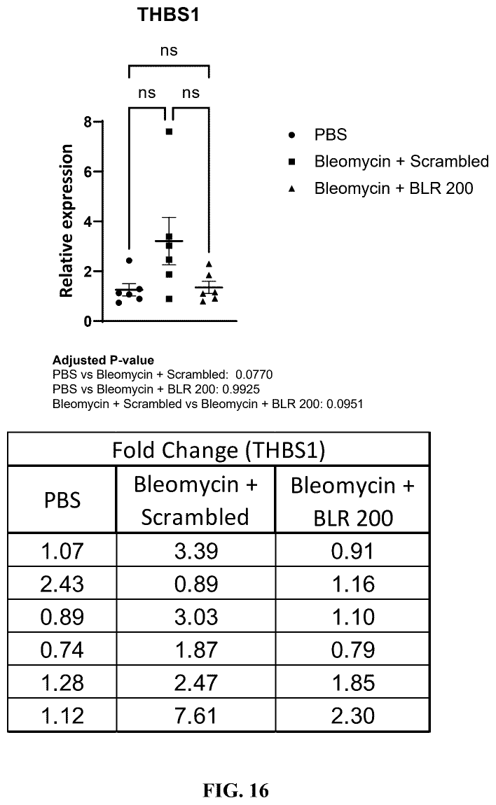

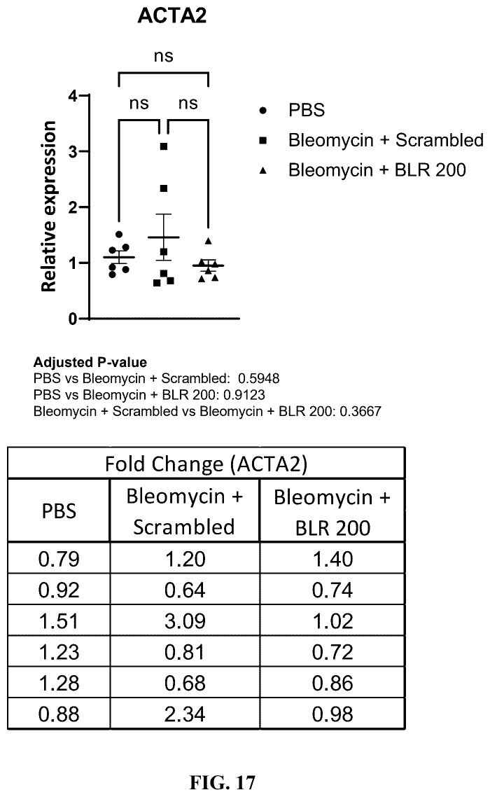

FIG. 16 is showing Thrombospondin-1 (THBS1) (also known as TSP-1, is a protein involved in various biological processes, particularly in inflammation, tissue repair, and fibrosis and activates TGF-b) lung mRNA levels are increased in IPF/lung fibrosis and decreased by BLR-200. FIG. 17 is showing ACTA2 (ACTA2 (Smooth Muscle Alpha-Actin, A-SMA) is a key protein marker of activated myofibroblasts, specialized cells responsible for depositing excessive extracellular matrix in fibrosis) lung levels are not affected in the diseased animals nor by BLR-200.

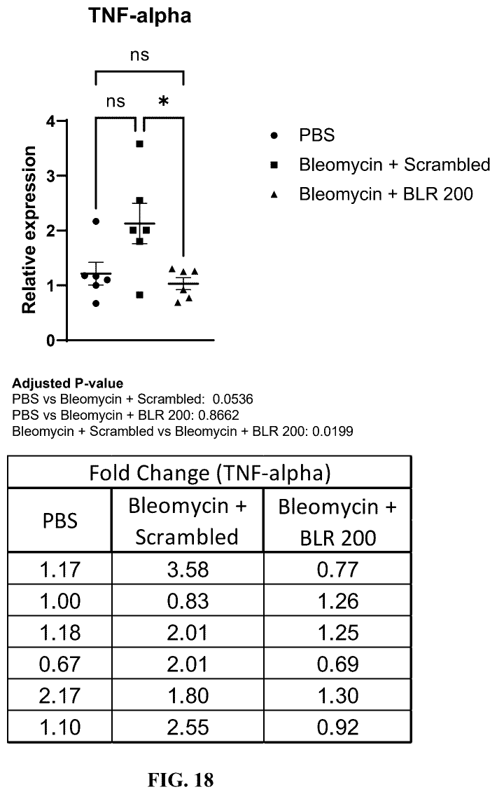

FIG. 18 is showing TNF-alpha (Tumor Necrosis Factor alpha, a cytokine that plays a crucial role in the immune system and is involved in both acute and chronic inflammation) mRNA levels are increased in IPF/lung fibrosis and normalized by BLR-200.

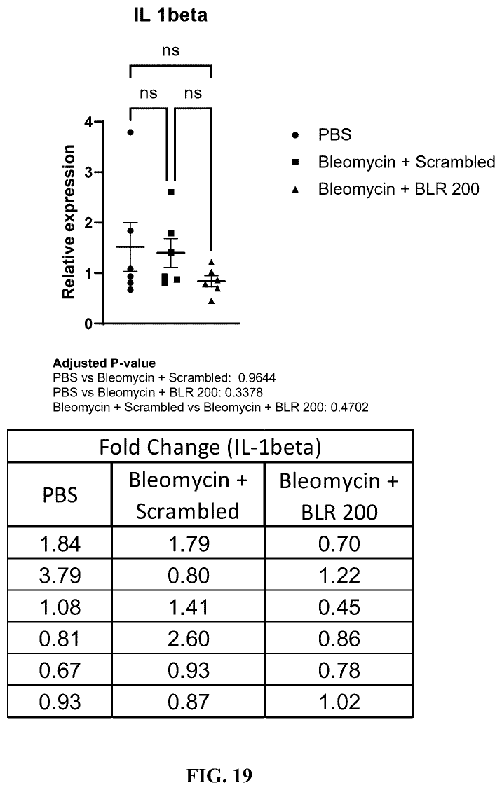

FIG. 19 is showing IL1beta (Interleukin-1 beta is a pro-inflammatory cytokine involved in the body's immune response to infection and injury) lung mRNA levels are not affected by IPF nor the BLR-200.

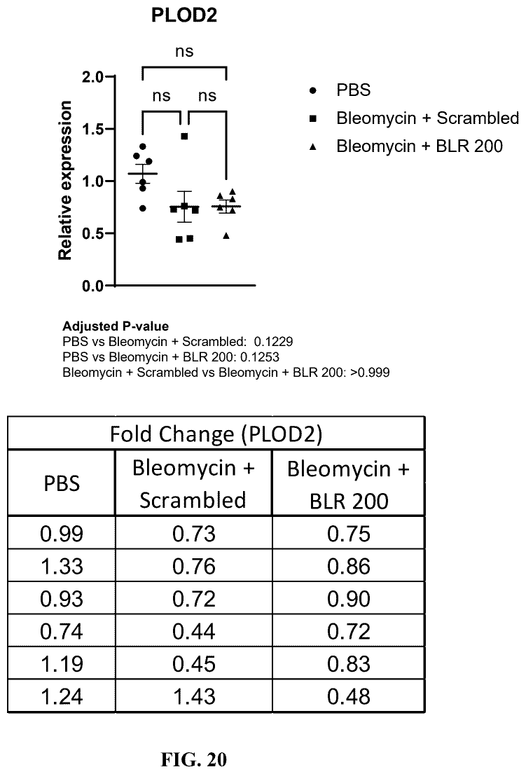

FIG. 20 is showing PLOD2 (Procollagen-lysine, 2-oxoglutarate 5-dioxygenase 2, an enzyme involved in collagen cross-linking, is upregulated in fibrotic tissues and contributes to matrix stiffening and collagen stabilization, thus fibrosis) lung mRNA levels were unexpectedly lowered in IPF and not affected by BLR-200.

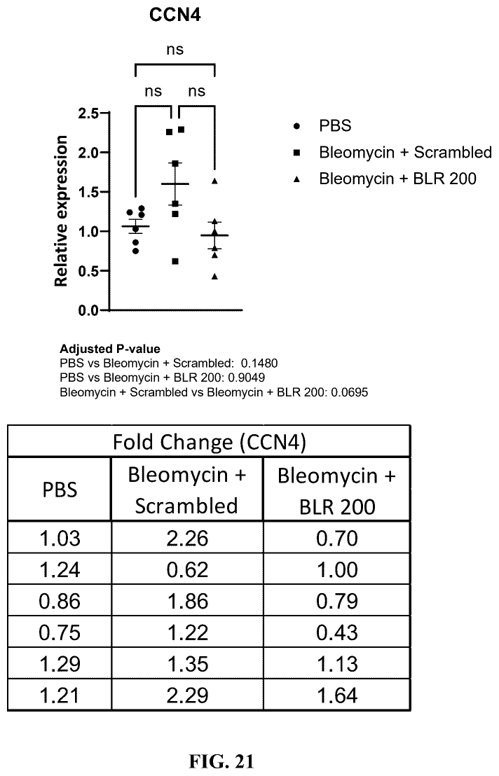

FIG. 21 is showing CCN4 (also known as WISP1, plays a role in promoting tissue fibrosis in IPF and lung fibrosis) lung mRNA levels Was elevated in IPF and normalized by drug treatment.

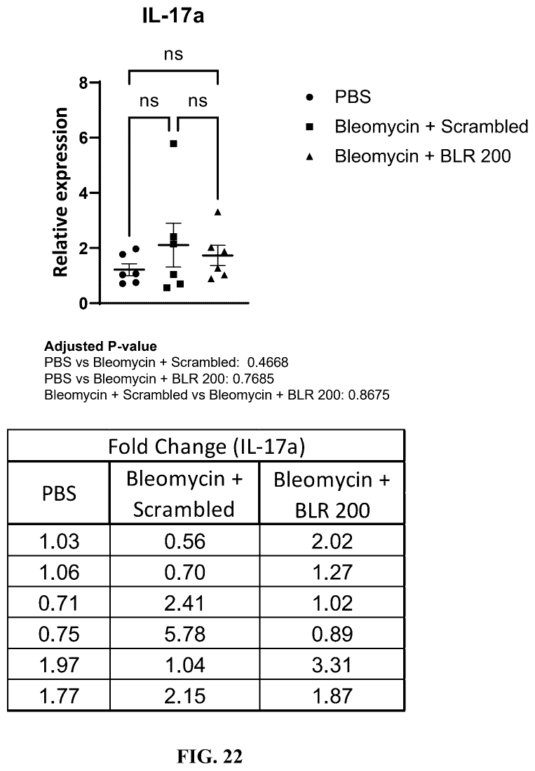

FIG. 22 is showing IL-17a is not elevated in IPF and not affected by drug treatment.

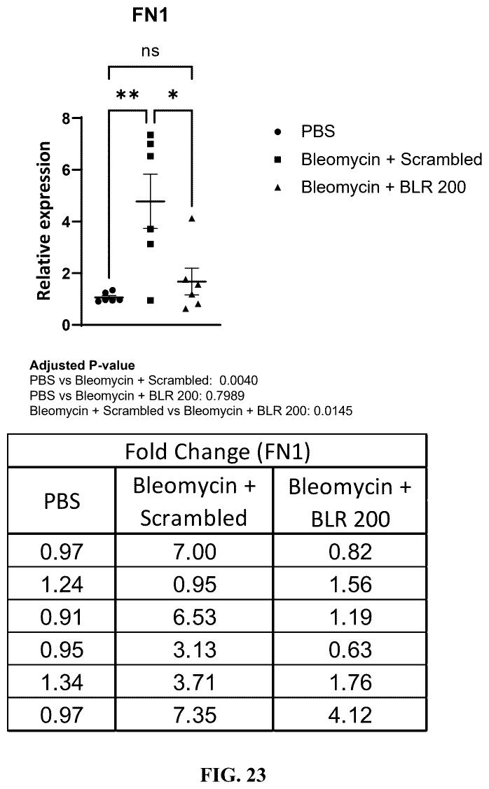

FIG. 23 is showing Fibronectin-1 (FN1), involved in a variety of cellular processes, including cell adhesion, migration, and the organization of the extracellular matrix and is implicated in the development of various diseases, including cancer, fibrosis, and cardiovascular disease, lung mRNA levels were increased in IPF and decreased by BLR-200 treatment.

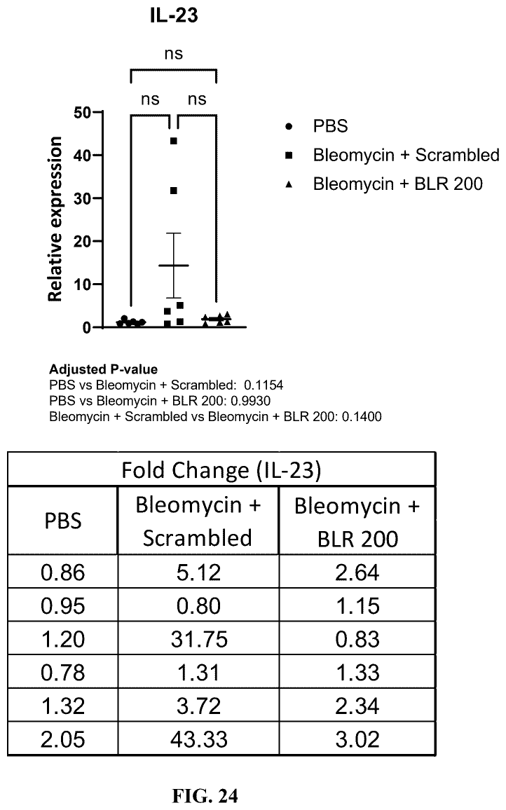

FIG. 24 is showing IL-23 lung mRNA levels were not affected by IPF nor the drug treatment.

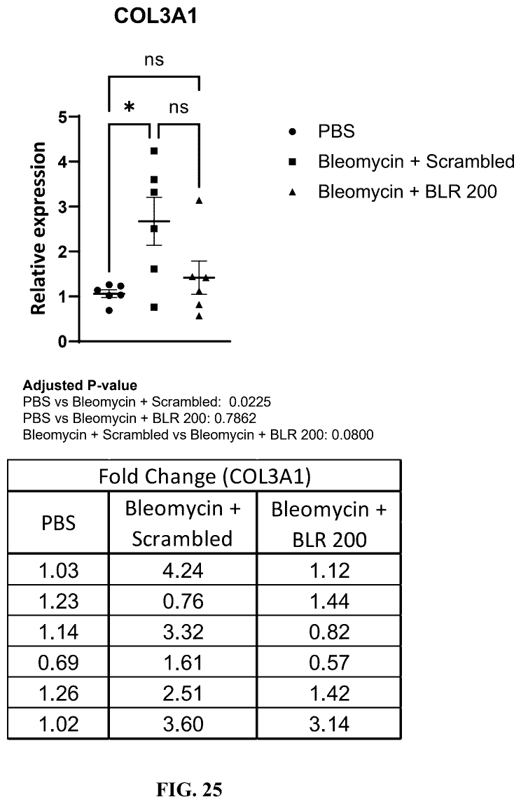

FIG. 25 is showing COL3A1 (crucial for the strength and support of various tissues, including skin, lungs, intestinal walls, and blood vessels) lung mRNA levels were increased in IPF and normalized by BLR-200.

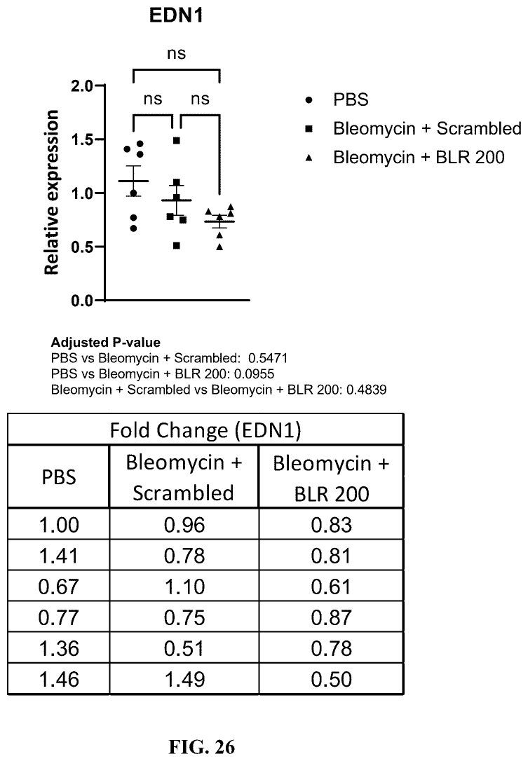

FIG. 26 is showing that Endothelin-1 (EDN1), that promotes myofibroblast induction through the ETA receptor via a Rac/phosphoinositide 3-kinase/Akt-dependent pathway and is thought to be essential for the enhanced contractile phenotype of fibrotic fibroblasts, lung mRNA levels were not affected by IPF nor BLR-200.

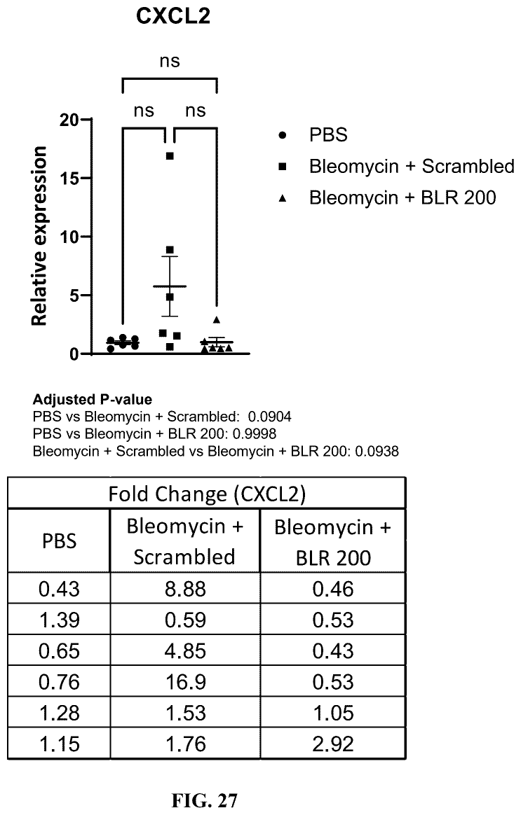

FIG. 27 is showing CXCL2 (when overexpressed is associated with exacerbated lung fibrosis in animals) lung mRNA levels were increased in some of the IPF animals, but none of the BLR-200 treated animals.

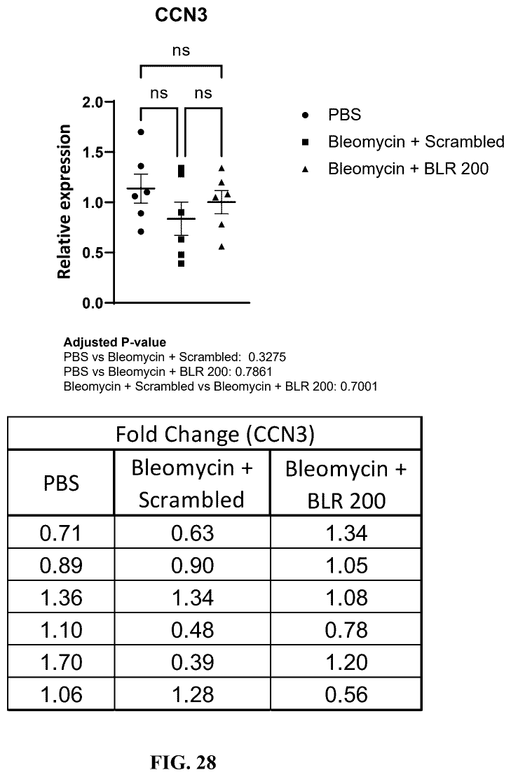

FIG. 28 is showing CCN3 (known to be antifibrotic in some types of fibrosis) lung mRNA expression appeared decreased as might be expected in IPF and levels appeared normalized in the BLR-200 treated mice.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating a patient with Idiopathic Pulmonary Fibrosis (IPF) and/or related lung fibro-inflammatory disease, including Interstitial Lung Disease, comprising the steps of: administering an effective dose of a synthetic peptide based on: 1) the IGFBP-like domain (insulin-like growth factor binding protein) contained in the CCN3 protein (Cellular Communication Network protein) or, 2) BLR-200 that is based on the structure/function of the IGFBP-like domain and with the amino acid sequence of SEQ ID NO. 1: SDRSADPSNQTGIS), or 3) structurally related sequences within the entire IGFBP-like domain, including SEQ ID NO. 2: GLYSDRSADPSNQTGIS, or SEQ ID NO. 3: CDRSADPSNQTGIC, or SEQ ID NO. 4: GLYCDRSADPSNQTGIC, 4) or a recombinant CCN3 protein containing the IGFBP domain-all peptides being either the native sequence, or with one or more amino acid substitutions where e.g. the cysteines substituted by serine.

2. The method of claim 1, wherein the patient is not diagnosed with IPF but has predisposition for developing IPF or lung fibrosis, to prevent IPF or lung fibrosis.

3. The method of claim 1, wherein the synthetic peptide is incorporated into a kit formulated in saline or phosphate balanced saline (PBS), or other such carrier, or the same freeze dried and in a vial that can be reconstituted in water or placed into a prefilled syringe, and then administered to a patient by a IV, IP, SC, IM injection, or in an oral delivery form, or into the lungs as an inhaled suspension via any of many lung delivery devices.

4. The method of claim 3, wherein a formulation is loaded into a device that is either implanted in the patient or is attached to the outside of the body and is designed to administer the drug.

5. A method of claim 3, where the drug is administered from about 1 time per week to about 4 times per week.

6. A method of claim 3, where drug dose administered is between about 0.081 μg/kg and about 1.62 μg/kg (based on the conversion to human of a mouse effective dose of between about 1 μg/kg and about 20 μg/kg.

7. A method of claim 6, where the drug dose is adjusted based of determination of the stage or condition of fibro-inflammatory progression/regression.

8. A method of claim 7, where the determination of drug dose is by the measurement of serum, tissue/body fluid, bronchial washings of biomarkers/markers, or CT scan MRI or similar diagnostic for fibro-inflammatory disease/fibrosis, and/or stage of IPF or SSc.

9. A diagnostic system for IPF/SSc lung fibro-inflammatory disease, comprising:
1. determining the stage of the IPF/SSc lung fibrosis and the early response to therapy by using measuring the change in response to BLR-200 in levels of markers, wherein the level of markers are obtained from at least on source including blood/plasma, lung tissue, or bronchiolar washings;
2, wherein the change in measurement is selected from the group consisting of: COL1A, COL3a, CCN1, CCN2, CCN3, CCN4, CXCL2, EDN1, FN1, IL-23, IL17A, PLOD2, IL-beta, TNF-alpha, ACTA2, THBS1, ITGA1, SOX2, IL33, TNC, PAI-1, MMP7, TGF-b1.

10. A diagnostic system of claim 9, where the overall stage determined can be used to adjust the level of medication to obtain the optimum response in the patient.

11. A diagnostic system of claim 9, where these measurements can by combined with CT scan MRI or similar to obtain a more accurate diagnostic and prognostic profile for fibro-inflammatory disease/fibrosis, and/or stage of IPF or SSc.

12. The method of claim 1, wherein the patient is diagnosed with IPF or is developing IPF or lung fibrosis, to prevent IPF or lung fibrosis.
